(19) United States
(12) Patent Application Publication
(10) Pub. No.: US 2019/0008906 A1
(43) Pub. Date: Jan. 10, 2019

(54) COMPOSITIONS FOR MANAGEMENT OF WOUNDS, SKIN DISEASES, DEHYDRATION, CHRONIC DISEASES, AND RESPIRATORY DISEASES

(71) Applicant: Noori AL-WAILI, Richmond Hill, NY (US)

(72) Inventor: Noori AL-WAILI, Richmond Hill, NY (US)

(21) Appl. No.: 15/916,586

(22) Filed: Mar. 9, 2018

Related U.S. Application Data
(60) Provisional application No. 62/529,615, filed on Jul. 7, 2017.

(51) Int. Cl. A61K 35/644; A61K 45/06; A61P 17/02; A61P 13/12

(52) U.S. Cl. CPC A61K 35/644; A61K 45/06; A61K 2300/00; A61P 13/12; A61P 17/02

(57) ABSTRACT

A composition including a mixture of at least two different forms of propolis is used to treat a variety of medical conditions. The composition can be administered topically, orally, by inhalation or parenterally. The composition is useful in treating wounds, infections, diabetes, skin conditions, renal conditions, dehydration, malnutrition, hypertension and various other conditions. It has been found that the mixture of at least two different types of propolis, either alone or in combination with other natural products is more effective than the administration of single propolis.

### CROSS-REFERENCE TO RELATED APPLICATIONS

This application claims priority under 35 USC 119(e) of U.S. Provisional Application No. 62/529,615, filed on Jul. 7, 2017, the disclosure of which is herein incorporated by reference.

### BACKGROUND OF THE INVENTION

### 1. Field of the Invention

The present invention relates to compositions for management of wounds, skin diseases, dehydration, chronic diseases, and respiratory diseases. In particular, the invention relates to using mixed propolis, either alone or in combination with other products for the treatment of various afflictions of the human body.

### 2. The Prior Art

It is well known that various bee products have healing properties and are suitable for treating wounds, skin diseases and other afflictions.

Propolis is a natural plant product collected by Apis mellifera honeybees from plants. It is a resinous mixture of botanical balsams and resin with digestive enzymes of bees. It contains more than 300 natural compounds including polyphenols, phenolic aldehydes, amino acids, steroids, sequiterpene-quinones, coumarins, and inorganic compounds. The chemical composition and biological properties of propolis depend on phytogeographical areas, seasonal collection time, and botanical sources. Propolis has no constant chemical composition. Depending on its region of origin, this substance consists of approximately 55% resinous and balm substances, approximately 30% wax, 5 to 10% essential oils, 2 to 5% pollen, vitamins and microelements. Therefore, mixing of propolis certainly will result in superpropolis. Propolis is relatively non-toxic, with a no-effect level in a 90-mouse study of 1400 mg/kg body weight/day (Burdock G., Review of the biological properties and toxicity of bee propolis (propolis). Food Chem Toxicol 1998;36: 347-363).

Propolis has antioxidant, immune-modulatory effects, antimicrobial, anti-inflammatory, antitumor, antiulcer, hepato and cardio-protective properties. Propolis also has antimicrobial activity and has renal and hepatic protection property.

CAPE (caffeic acid phenethyl ester) is an active phenolic part of propolis and has antitumoral, antiproliferative, anti-inflammatory, antineoplastic, and antioxidant properties. The molecular weight and empirical formula of CAPE are 284.3 g/mol and C₁₇H₁₆O₄. All flavonoids within propolis, but not CAPE, are reported to have a low order of acute oral toxicity with a reported LD50 of 8-40 g/kg. Similarly, a safe dose in humans is estimated as 1.4 mg/kg body weight/day, or approximately 70 mg/day (Burdock G., Review of the biological properties and toxicity of bee propolis (propolis). Food Chem Toxicol 1998; 36: 347-363).

Honey has served as a common base for wound healing compositions since ancient times. Honey as a healing composition was mentioned in the Talmud, both the old and new testaments of the Bible, and the Holy Quran. Honey has been found to have antibacterial, antifungal, antiviral, anti-inflammatory, antihypertensive, antioxidant, antitumor, cardioprotective, hepatoprotective, and hypoglycemic properties. It has been found that there is a large amount of variation in potency of antibacterial activity between different honeys, most likely due to varying levels of antibacterial factors in honey.

Garlic (Allium sativum L.) is one of the world's oldest medicines and it has biological activities, including anti-carcinogenic, antiatherosclerotic, antithrombotic, antimicrobial, antiinflammatory and antioxidant effects.

Curcumin belongs to the family of natural compounds collectively called curcuminoids and it possesses remarkable beneficial anti-oxidant, anti-inflammatory, anti-cancer, and neuroprotective properties. It is the most active component of rhizome of Curcuma longa L. (common name: turmeric). Furthermore, curcumin is effective in the treatment of chronic pain, inflammatory dermatoses, acceleration of wound closure, skin infections, as well as dyspigmentation.

Olive oil contains 95 to 99% acyl glyceroles, 0.5 to 1.5% unsaponifyable substances and 0.1 to 3% free fatty acids, glycerides, secciridoids and flavonoids. The main fatty acids are oleic acid, palmitic acid and linoleic acid. Furthermore, it contains phenolic compounds, carbohydrates, steroles, triterpene alcohols, hydroxy triterpenic acids, tocopheroles, phospholipids, carotinoids, chlorophyll and pheophytines.

Nigella sativa belongs to the botanical family of Ranunculaceae and commonly grows in the Eastern Europe, Middle East, and Western Asia. It is a small shrub with tapering green leaves and rosaceous white and purplish flowers. Its ripe fruit contains tiny seeds, dark black in color. Nigella sativa is native to Southern Europe, North Africa and Southwest Asia, Middle Eastern Mediterranean region, India, Pakistan, Syria, and Turkey. Among Muslims, it is considered as one of the greatest forms of healing medicine available as was mentioned that black seed is the remedy for all diseases except death in one of the Prophetic hadith. It is also recommended for use on regular basis in Tibb-e-Nabwi (Prophetic Medicine). Nigella sativa seeds contain an essential oil (0.4-2.5%), fixed oil (36-48%), alkaloids, saponin and proteins. Nigella sativa oil was shown to contain thymoquinone which is the main bioactive component (27.8-57.0%) of the essential oil of the black seed. Toxicity studies on laboratory animals have reported that nigella sativa oil and thymoquinone are quite safe, mainly when given orally.

Data has showed that nigella sativa has diuretic, antihypertensive, antidiabetic, anticancer and immunomodulatory, analgesic, antimicrobial, anthelmintics, analgesics and anti-inflammatory, spasmolytic, bronchodilator, gastroprotective, hepatoprotective, renal protective and antioxidant properties. The seeds of nigella sativa are widely used in the treatment of various diseases like bronchitis, asthma, diarrhea, rheumatism and skin disorders. It is also used as liver tonic, digestive, anti-diarrheal, appetite stimulant, to increase milk production in nursing mothers to fight parasitic infections, and to support immune system. Nigella sativa oil and thymoquinone have been used as anti-inflammatory, antioxidant and anticancer therapeutic Agents.

Bee venom therapy is thousands of years old and it involves the application of live bee stings to the skin or the injection of bee venom into the skin. Bee venom is produced by honeybees (Apis mellifera). Bee venom has anti-inflammatory, antibacterial, and anti-rheumatic activities. It has been used in arthritis, rheumatism, back pain, multiple sclerosis, cancer, and skin diseases. It relieves pain and inflammation and it has an immune response enhancing effect. It contains melittin, apamin, adolapin, phospholipase A2, mast cell degranulating peptide, α-D-glucosidase, biologically activity amines, hyaluronidase, acid phosphomonesterase, and lysophospholipase. Melittin has anti-inflammatory and antioxidant activity.

Syzygium aromaticum (synonym: Eugenia cariophylata) is commonly known as clove. It is a medium sized tree (8-12 m) native from the Maluku islands in east Indonesia. Cloves are the aromatic flower buds of a tree in the family Myrtaceae. Clove represents one of the major vegetal sources of phenolic compounds as flavonoids, hidroxibenzoic acids, hidroxicinamic acids and hidroxiphenyl propens. Eugenol is the main bioactive compound of clove. Other phenolic acids found in clove are the caffeic, ferulic, elagic and salicylic acids. Flavonoids as kaempferol, quercetin and its derivates (glycosilated) are also found in clove in lower concentrations. Concentrations up to 18% of essential oil can be found in the clove flower buds. It was estimated that 89% of the clove essential oil is eugenol and 5% to 15% is eugenol acetate and β-cariofileno.

The essential oil extracted from flower buds of clove is used as a topical application to relieve pain and to promote healing. Its main constituents are phenylpropanoids such as carvacrol, thymol, eugenol and cinnamaldehyde. In addition to its antimicrobial, antioxidant, antifungal and antiviral activity (herpes simplex), clove essential oil possesses anti-inflammatory, cytotoxic, insect repellent and anesthetic properties. Syzygium aromaticum (clove) has an anti-microbial activity.

Acacia Arabica gum is a traditional oral hygiene substance that has been used for centuries by many communities in the Middle East and North Africa. It consists mainly of Arabica, a complex mixture of the calcium, magnesium and potassium salts of Arabic acid. There are also other constituents such as tannins, cyanogenic glycosides, oxidases, peroxidases and pectinases; all of which have been shown individually to exhibit antimicrobial properties.

Gum Arabic is mixture of polysaccharides, oligosaccharides and glycoproteins. It is exudates of Acacia Senegal/seyal trees. It is water soluble; therefore, it is used as an emulsifier, thickening substance and flavor stabilizer in many pharmaceutical and food industries.

Bees collect pollen from plant anthers, mix it with a small dose of the secretion from salivary glands or nectar, and place it in corbiculae which are situated on the tibia of their hind legs. The chemical composition of bee pollen depends strongly on the plant source and geographic origin, together with other factors such as climatic conditions, soil type, and bees' race and activities. Bee pollen contains about 250 substances including amino acids, lipids (triglycerides, phospholipids), protein, vitamins, macro- and micronutrients, phenolic compounds, enzymes, and coenzymes and flavonoids.

Bee pollen contains 22.7% of protein on average (10.4% of essential amino acids, significant amounts of nucleic acids), digestible carbohydrates (30.8% on average), essential fatty acids, phenolic compounds (flavonoids, leukotrienes, catechins, and phenolic acids), multivitamins 0.7%, calcium, phosphorus, magnesium, sodium, and potassium, iron, copper, zinc, manganese, silicon, and selenium 1.6%. Bee pollen has antifungal, antimicrobial, antiviral, anti-inflammatory, immune-stimulating, and local analgesic and also facilitates the granulation process of the burn wound healing.

Royal jelly is a mixture of yellow-white creamy and acidic secretions produced by the worker honeybees using their mandibular and hypopharingeal glands to supply food for a queen honeybee. It is rich in protein, amino acids, fatty acids, calcium, iron, minerals, vitamins, and carbohydrates. 10-HAD is the most active ingredient. Royal jelly has antimicrobial properties against yeast, Gram-negative and Gram-positive bacteria. Royal jelly stimulates immune-co-potent cell proliferation and production of antibodies in mice.

Flaxseed (linseed, Linum usitatissimum), is the richest plant source of α-linolenic acid (50%-62% of flaxseed oil, or 22% of whole flaxseed) and lignans (a class of phytoestrogen, range: 0.2-13.3 mg/g flaxseed) and it contains dietary fiber (28% by weight), a third of which is soluble fiber. Whole flaxseed or flaxseed oil are included in the general oral composition and renal healing composition.

Juglans regia Linn. (Juglandaceae) is a valuable medicinal plant and various parts of the plant have been used for management of diseases in different cultures. Walnuts contain unsaturated fatty acids, proteins, fiber, vitamins, minerals, phytosterols and polyphenols. Furthermore, walnuts contain a number of potentially neuroprotective compounds like vitamin E, folate, melatonin, several antioxidative polyphenols and significant amounts of ω-3 fatty acids. Its health benefits include reduced risk of cardiovascular disease, certain cancer and type II diabetes, and amelioration of symptoms of neurological disorders. It is rich in polyphenols and other phytochemicals, polyunsaturated and fatty acids with a particularly high ω3:ω6 ratio—the highest among all the tree nuts.

While each of these natural ingredients alone have healing properties, it would be desirable to produce a product that increases the effectiveness of the natural ingredients beyond what has already been documented.

### SUMMARY OF THE INVENTION

It is an object of the invention to provide a healing substance that exhibits superior healing properties as compared to the natural products alone described above.

This object is accomplished by a product that contains a mixture of different types of the same substance, namely propolis. By mixing different propolis varieties, the various beneficial properties of the different propolis types are combined and enhance the healing process.

The propolis can be mixed in any desired proportions, such as 1:1 ratio (generally 45-55 wt % each). Other ratios could also be used.

The mixed propolis formulations are useful for the prevention/treatment of, inter alia, wounds, ulcers, burns, skin infection, and related skin disorders such as contact dermatitis, eczema, psoriasis, wrinkle and other skin diseases (inflammatory, infectious or allergic). The compositions are also antimicrobial to fungus, mold, bacteria, viruses and yeasts, indicating potential to treat skin, wound and ulcer infections in human or animals.

The invention also comprises oral compositions for treatment of, inter alia, anemia, enhancement of immunity, potentiating the effects of antibiotics and chemotherapy, for management of dyslipidemia, diabetes mellitus, cardiovascular diseases, H. pylori gastritis or ulcers, dyspepsia, parasitic diseases, acute and chronic kidney diseases and hypertension, and for prevention or amelioration of toxic effects of heavy metals or chemotherapies or radio therapy.

The invention also comprises intravenous compositions for protection of, inter alia, liver, kidney, bone marrow when used with chemotherapy, cytotoxic drugs, and immunosuppressive agents, or used in inflammatory diseases, infectious diseases or sepsis or dehydration or malnutrition or part of cancer management.

The invention also comprises inhalation compositions for the management of infectious respiratory diseases, sinusitis, and lung obstructive diseases or malignancies.

Although honey, propolis, royal jelly and bee pollen alone or mixed with other substances are already known in the medical science, yet so far mixtures of different propolis types has never been used in topical, parenteral, inhalational or oral treatment.

Propolis has no constant chemical composition, which is dependent on the region of origin, seasons, and plant origin. The functional properties therefore are different in their potency. The different compositions and properties can be utilized further by mixing samples of propolis collected from different regions or plant source to provide new propolis with higher nutritional values and more potent medicinal applications.

The objective of the present invention is to provide a natural agent for nutritional and medicinal use, which has a broad application in human and animals, which also composed of multiple natural ingredients and, therefore, is lack of adverse effects, without contraindication, and well tolerated.

A further objective of the present invention is to use the natural ingredients in doses, which are suitable for human or animal use and without side effect.

A particular advantageous use of the mixed propolis, alone or in combination with one or more than one of the following natural substances such as honey, bee pollen or mixed pollen, royal jelly or mixed royal jelly, nigella sativa, thymoquinone, CAPE, curcumin, bee venom or melittin, garlic or aged garlic or aged garlic extract (AGE), flaxseed or flaxseed oil, curcumin, green tea, fenugreek seeds, walnuts, ginger, acacia Arabica, clove or olive oil will provide a composition with high potency, wide spectrum of medicinal and nutritional use, low cost, lack of adverse effects, well tolerability, ability to potentiate the use of chemotherapy, antibiotics and other modern interventions, improving circulation, and ameliorating side effects of immunosuppressive and chemotherapeutic agents as well as radiotherapy.

A further advantageous use of the substance mixture according to the invention is that treatment with these substances includes a decrease in length of hospital stay, decrease in complications of chronic diseases such as diabetic nephropathy, diabetic retinopathy, hypertensive heart disease, burn scars and deformities, wound scars, and decreasing further invasive interventions.

A further advantageous use of the substance mixture according to the invention consists in preparing compositions for, not limited to, skin care, skin diseases, diabetes mellitus, hypertension, cardiovascular diseases, kidney diseases, urinary calculus, infections (bacterial, viral and parasitic), malignancies, malnutrition, and respiratory diseases.

A further advantageous use of the substance mixture according to the invention consists in preparing compositions for to be used in topical, parenteral, inhalational or oral treatment.

Another advantageous use of the substance mixture according to the invention includes providing biological systems with potent antioxidant capacity, potent antimicrobial intervention, potent anti-inflammatory agents, potent analgesic intervention, and potent nutritional support more than using individual ingredients. There is no intervention in modern medicine available with all these properties together.

Further, it is an object of the present invention to provide a composition with a base of mixed propolis, which is suitable for the treatment of parasitic diseases such as Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total propolis or propolis extract, such as water, alcohol or ethyl acetate extracts.

The different compositions can be administered topically, orally, parenterally, by inhalation (nasal or oral), vaginally, or rectally in dosage unit formulations containing conventional nontoxic pharmaceutically acceptable carriers, adjuvants, and vehicles, as desired. The term parenteral as used herein includes intravenous, intramuscular, intraperitoneal, intrasternal injection, or infusion techniques.

The wound/skin healer composition is a composition for topical application in or on a wound or ulcer that is believed to promote healing and tissue growth, provide nutritional factors and antioxidants, and to eradicate infection and accelerate wound healing. The composition includes mixed propolis, either alone or combined with, honey, CAPE, bee venom or melittin, bee pollen or mixed bee pollen, royal jelly or mixed royal jelly, curcumin, acacia Arabic, olive oil, Nigella Sativa or thymoquinone, clove, and garlic or aged garlic or AGE. Acacia Arabica is used in heavy exudate, bleeding wounds, and also to increase viscosity of the formulae. It has antioxidant and antimicrobial effects. Multiple compositions can be prepared with mixing any two or more ingredients including propolis to achieve the same goals.

The powders of crude ingredients or their extracts are mixed into the viscous honey base or any acceptable base, and the composition may be applied directly to the wound or ulcer or skin diseases, or the composition may be applied to sterile gauze or incorporated in any wound materials such as alginate, foams, hydrocolloids, interactive dressing, or composite dressing and the wound or ulcer, particularly an open surgical wound or deep ulcer, may be packed with the gauze.

The amount of the wound/skin compositions to be administered and the frequency of administration and period of the treatment depends on the type, location, chronicity, and severity of the wound, ulcer or infection. The compositions may be presented in unit-dose or multi-dose containers. And they may include additional ingredients that are conventional in the art with regard to topical formulations. The healing herein includes curing, alleviating, reversing, palliative and prophylactic treatment of the condition.

The main advantage of the mixed propolis composition according to the invention includes:

1—Natural products and available everywhere,

2—Safe and almost no side effects,

3—Synergistic effects as a wound healer as well as antimicrobial to eradicate infections,

4—Provides wound and skin with excellent nutritional, anti-inflammatory and antioxidants ingredients, and

5—Debridement of dead tissue in wounds and ulcers.

The products are for wound and ulcer treatment, which consist of multi component combination of natural substances. This composition allows batch process production, where it is possible to accomplish mixing and fusion of various natural substances, and cream homogenization, by converting all substances in the dissolvable stage. Honey is used as a semiliquid and can be combined with other ingredients. Hard propolis is converted into dissolvable stage by extraction with ethanol, obtain water extract, or could be used as ground powder. Bee pollen powder is dried and ground into powder. Other ingredients are used as ground powder, or water and alcohol extracts or as an oil form.

One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, wax, beeswax, and the like.

Gels, ointments and creams may be formulated, for example, with an aqueous or oily base with the addition of suitable thickening. Lotions may be formulated with an oily or aqueous base and can contain one or more emulsifying agents, dispersing agents, stabilizing agents, coloring agents, and/or suspending agents and thickening agents.

Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA.

The composition can be applied directly to the wound as a gel, ointment, liquid, cream, or the like as described above. Alternatively, the composition is administered in the form of a wound dressing. The wound dressing may include any one of the different types of substrates and/or backings that are commercially available, including films such as polyurethane films, hydrocolloids such as hydrophilic colloidal particles bound to polyurethane foam, hydrogels, foams such as hydrophilic or hydrophobic, calcium alginates, and cellophane. For example, the composition can be applied to the surface of, or incorporated into a dressing gauze or matrix. Suitable gauze dressings may include, for example, a swabs, dry woven or non-woven sponges, bandages and wraps with varying degrees of absorbency. Exemplary fabric compositions may include cotton, rayon or polyester. In certain embodiments, gauzes and non-woven dressings may be available sterile and with or without an adhesive border. In certain embodiments, the dressings also comprise one or more additional pharmaceutically active compounds and/or carrier agents, such as xeroform, oil, saline, zinc salts, petrolatum, and scarlet red. The agent to be used according to the invention may be prepared in all the application forms common for topical medicaments, such as an ointment, shampoo emulsion, stocking, sponge, soap, facial pack, lotion, bath salts, or plaster.

The present composition and/or the components of the composition may be sterilized by any suitable method, including conventional, well-known sterilization techniques, such as autoclaving. Raw, unheated honey can be sterilized, for example, by ozonizing the honey.

The dosage regimen for treating wounds is selected in accordance with a variety of factors including the age, weight, sex, and medical condition of the patient, the severity of the wound, the route of administration, pharmacological considerations such as the activity, efficacy, pharmacokinetic and toxicology profiles of the particular composition used, whether a dressing or drug delivery system is used and whether the composition is administered as part of a drug combination.

The advantageous use of the substance mixture according to the invention consists in preparing an agent for wound and ulcer healing. These properties include antioxidant, anti-inflammatory, antibacterial, and antimycotic effects; wound and ulcer healing activity; supplying vitamins, trace elements, natural glucose, and amino acids. The mixture will provide stronger antioxidants, anti-inflammatory and antimicrobial activities as compared to single ingredient treatments.

The oily content of the compound acts as a barrier, preventing excessive loss of body fluids and decreasing evaporative wound water loss thus preventing wound desiccation and providing optimal wet atmosphere for wound healing. Olive oil contains polyphenolic compounds, which has an antibacterial effect to enhance wound healing. The compositions may include deodorized garlic (Allium sativum), such as that described in (U.S. Pat. No. 4,933,201 to Sakai) or pure, powered, pulverized, minced or extract, preferably obtained from the bulb, but not limited to any portion of the plant, preferably derived from the aforementioned member of the genus, but not limited to any species within the Allium genus such as Allium oleraceum, Allium ursinum, Allium ampeloprasum and Allium canadense. The compositions may include pure, solids, decolorized or extract of Turmeric Rhizome (Curcuma Longa), preferably derived from this member of the genus, but not limited to variation in species of the curcuma genus such as Curcuma aromatica and Curcuma amada.

Suggested compositions for treatment of wounds, ulcers, and skin diseases are illustrated in, not limited to, tables 3-6.

The invention also relates to a herpes simplex 1 and 2 and zoster healing composition that comprises the topical use of mixed propolis with or without honey or or CAPE, curcumin, bee venom or melittin, royal jelly or mixed royal jelly, and nigella sativa or thymoquinone in form of gel, ointment or cream. The mixed propolis can be used alone or in combination with one or more than one of the ingredients of the composition. The composition is applied directly on the affected area 3-6 times per day or according to the severity of the lesions. The recipe might include ingredients that help dissolving propolis in water. The healing herein includes curing, alleviating, reversing, palliative and prophylactic treatment of the condition. The composition should be used alone or in combination with drugs used in the management of herpes infection. Multiple compositions can be prepared with mixing any two or more ingredients including mixed propolis to achieve the same goals. The formulations can include total propolis or propolis extract, such as water, alcohol or ethyl acetate extracts.

This composition allows batch process production, where mixing and fusion of various natural substances, and cream homogenization are possible, by converting all substances in dissolvable stage. Hard propolis is converted into the dissolvable stage by extraction with ethanol or ethyl acetate, is obtained as a water extract or could be used as ground powder. Bee pollen is dried and ground into powder. Honey is used as a semiliquid. Other ingredients are used as ground powder, or water and alcohol extracts or as an oil form. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, wax, beeswax, and the like. Gels, ointments and creams may be formulated, for example, with an aqueous or oily base with the addition of suitable thickening. Lotions may be formulated with an oily or aqueous base and can also contain one or more emulsifying agents, dispersing agents, stabilizing agents, coloring agents, and/or suspending agents thickening agents. Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA. The composition can be applied directly to the wound as a gel, ointment, liquid, cream, or the like as described above.

The main advantages include;

1—Natural products and available everywhere;

2—Safe and almost no side effects;

3—Synergistic effects as a herpes simplex and zoster healer as well as an antimicrobial to eradicate bacterial and fungal infections, and

4—Provides skin with excellent nutritional, anti-inflammatory and antioxidants ingredients

A suggested composition for treatment of herpes simplex and zosters is illustrated in, not limited to, table 7.

The intravenous administration of mixed propolis extracts or CAPE with or without honey or honey extracts, thymoquinone or curcumin or curcumin extract can be used alone or sequentially or simultaneously with the intravenous antibiotics or cytotoxic agents. Other aspects of the invention relate to the use of mixed propolis, CAPE as suitable forms for parenteral administration.

The intravenous mixed propolis is for protection of, not limited to, liver, kidney, bone marrow or any other body organs or tissues when used with antibiotics, cytotoxic drugs, and immunosuppressive agents or used in inflammatory diseases, infectious diseases or sepsis.

The amount of the compositions to be administered, the rate of administration and the time of administration will depend on the type of infection to be treated, the antibiotic or cytotoxic agent to be used, and the size, location, progression and/or severity of the infection to be treated. Appropriate dosage for antibiotics or cytotoxic agents, are well known in the art. In some embodiments, mixed propolis or mixed propolis extract with or without honey is infused daily or every other day or more than one time daily until antibiotic or cytotoxic agent course is finished. Extension of the propolis composition infusion might be considered in such case of recurrent infections or malnutrition of severe infection or sepsis.

The methods of the present application are useful for the treatment of various infectious diseases, such as bacterial infection, viral infection, fungal infection or parasitic infection, systemic or local.

The invention will be illustrated below with the following example, which is in no way restrictive.
Mixed propolis extract: 0.1-30 mg/kg.b.wt
CAPE: 0.001-10 mg/kg.b.wt or 0.1-500 micromole/kg.b.wt
Thymoquinone: 0.001-5 mg/kg.b.wt
Curcumin or curcumin extract: 0.1-40 mg/kg
Honey or water, ethanol or ethyl acetate extract: 0.1-2 g/k.b.wt

The compositions may be presented in unit-dose or multi-dose containers. And they may include additional ingredients that are conventional in the art with injectable formulations. The healing herein includes curing, alleviating, reversing, palliative and prophylactic treatment of the condition. Water soluble propolis extract was prepared or using the method described elsewhere (Nikolov N., et al., Bulgarian J Pathology Applied, 1987; 79903/28, 05).

The aims of the composition are:

1—Synergistic effect with antibiotics for treatment of local or systemic infections;

2—For treatment of acute kidney injury and acute liver injury,

3—Hydration,

4—Nutrition supplement, and

5—To decrease the side effects of immunosuppressive agents, cytotoxic agents, or antibiotics.

The invention also relates to a peritonitis healing composition, that comprises mixed propolis alone or with honey or CAPE, nigella sativa oil, and/or thymoquinone in the form of a recipe suitable for parenteral administration. It can be used alone, sequentially, or simultaneously with intravenous antibiotics. When administered simultaneously, the mixed propolis with or without honey or CAPE, or nigella sativa oil, or thymoquinone, or all ingredients together, can be in the same or separate compositions. The composition is used for management of acute or chronic bacterial, viral, fungal or parasitic peritonitis. It could be used in liver cirrhosis. The compositions might include any two of the ingredients or all the ingredients together. The compositions might be used alone or in combination with antibiotics. Mixed propolis alone can be used alone for the same healing purposes. The formulations can include total propolis or propolis extract, such as water, alcohol or ethyl acetate extracts.

The amount of the compositions to be administered, the rate of administration and the time of administration will depend on the type of infection to be treated, progression and/or severity of the infection to be treated. The composition is infused daily or every other day or more than one time daily. Extension of the composition infusion or injection might be considered in such case of recurrent infections or malnutrition or severe infection or sepsis.

The main advantages include;

1—Natural products and available everywhere;

2—Safe and almost no side effects;

3—Synergistic effect as an antimicrobial to eradicate infections; and

4—Provides the peritoneum with excellent nutritional and anti-inflammatory and antioxidant ingredients.

A suggested composition for treatment of peritoneal diseases is illustrated in, not limited to, table 8.

The general oral healing composition includes mixed propolis alone or combined with bee pollen or mixed bee pollen, royal jelly, flaxseed, acacia Arabica, curcumin, Nigella sativa or thymoquinone, garlic or aged garlic or AGE, olive oil, cloves, honey or CAPE, for treatment of, not limited to, anemia, enhancement of immunity, potentiating the effect of antibiotics and chemotherapy, for management of dyslipidemia, diabetes mellitus, hypertension, and for prevention or amelioration of toxic effects of heavy metals or chemotherapies. Multiple compositions can be prepared with mixing any two or more ingredients including propolis to achieve the same goals.

The oral healing composition is a product for oral use to maintain or potentiate the anti-oxidant levels in healthy individuals or in patient during treatment with a chemotherapeutic agent, antibiotics or pain medications such as NSAIDs.

Other aspects of the invention also relate to methods to reduce the toxicity of exposure of antibiotics or cytotoxic agents in a patient, and heavy metals in a patient or healthy individual comprising administering the healing oral formulae to the patient or normal individual with such exposures.

An important aspect relates to a method of reducing the dose of antibiotic or cytotoxic agents administered to a patient for purpose of management of infection, sepsis or malignancies, due to synergistic effects, anti-inflammatory, antioxidant properties and enhancement of immunity, comprising administering the healing oral compositions to the patient.

The compositions may be presented in unit-dose or multi-dose containers. And they may include additional ingredients that are conventional in the art with regard to oral formulations. The healing herein includes curing, alleviating, reversing, palliative and prophylactic treatment of the condition.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the mixture according to the invention consists in preparing an agent for increasing immunity, antioxidant capacity and healing cellular power, and also provides an excellent nutritional support in normal individuals and in patients with chronic diseases and infections. The mixture will provide stronger antioxidants, anti-inflammatory and antimicrobial than individual ingredient. These ingredients have anti-cancer properties and immune system stimulating activity.

The main advantages include:

1—Natural products and available everywhere,

2—Safe and almost no side effects,

3—Synergistic effects as a disease healer as well as an antimicrobial to eradicate infections, and

4—Provides all body system with excellent nutritional, anti-inflammatory and antioxidants ingredients.

A suggested composition for treatment of general medical diseases is illustrated in, not limited to, table 9.

The invention also relates to a renal healing composition, wherein the mixed propolis or extracts thereof is mixed with honey or extracts thereof or CAPE, flaxseed, acacia Arabica, curcumin or curcumin extract, bee pollen or mixed bee pollen, garlic or aged garlic or AGE or garlic oil, Nigella sativa or nigella sativa oil or thymoquinone for management of, not limited to, kidney diseases, proteinuria and urinary calculus, and is used as an oral recipe. The dose depends on the stage of the kidney disease, severity of proteinuria, etiology of proteinuria and the comorbidities. The composition presumably alleviates or cures proteinuria, and prevents kidney stone formation and facilitates urinary calculus passage. This composition provides a diuretic effect, antioxidants, anti-inflammatory action and analgesic effect. Multiple compositions can be prepared with mixing any two or more ingredients including propolis to achieve the same goals. The formulations can include total propolis or propolis extract, such as water, alcohol or ethyl acetate extracts.

The compositions may be presented in unit-dose or multi-dose containers. And they may include additional ingredients that are conventional in the art with regard to oral formulations. The healing herein includes curing, alleviating, reversing, palliative and prophylactic treatment of the condition. The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, beeswax, and the like. The advantageous use of the substance mixture according to the invention consists in preparing the product or composition that will be useful for management of, inter alia, proteinuria due to diabetes, nephritis, nephrotic syndrome, or due to any reason such as heavy metal intoxication, vasculitis, systemic lupus, IgA nephropathy, membranous nephropathy and others causes mentioned in the medical literature.

The mixture will provide stronger antioxidants, anti-inflammatory and antimicrobial than individual ingredients. The treatment goals of this mixture are:

1—healing of proteinuria,

2—healing of kidney injury in acute and chronic kidney diseases, and

3—as part of management of kidney and urinary calculus and crystaluria.

The main advantages include:

1—Natural products and available everywhere,

2—Safe and almost no side effects,

3—Synergistic effects as kidney healer as well as antimicrobial to eradicate infections, and

4—Provides urinary system with excellent nutritional, anti-inflammatory and antioxidants ingredients.

A suggested composition for treatment of kidney and urinary system diseases is illustrated in, not limited to, table 10.

The invention also relates to a H. pylori healing composition comprising mixed propolis alone or in combination with honey, CAPE, acacia Arabica, garlic, aged garlic or AGE or garlic oil, bee pollen or mixed bee pollen, olive oil, curcumin or curcumin extract, fish oil and cloves or cloves extract in form of oral recipe for management of H. pylori infection. The formulae could be used alone or in combination with antibiotics and a pump inhibitor. The healing herein includes curing, alleviating, reversing, palliative and prophylactic treatment of the condition. The compositions may be presented in unit-dose or multi-dose containers. And they may include additional ingredients that are conventional in the art with oral formulations. The formulations can include total propolis or propolis extract, such as water, alcohol or ethyl acetate extracts.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful to eradicate H. pylori infection, peptic ulcer and dyspepsia. Multiple compositions can be prepared with mixing any two or more ingredients including propolis to achieve the same goals.

The main advantages include:

1—Natural products and available everywhere;

2—Safe and almost no side effects;

3—Synergistic effects to heal H. pylori infection; and

4—Provides the gastrointestinal tract with excellent nutritional, anti-inflammatory and antioxidants ingredients.

A suggested composition for treatment of gastritis and H. pylori infection is illustrated in, not limited to, tables 11.

The invention also relates to a Clostridia difficile colitis healing composition that comprises the composition of mixed propolis alone or with honey, CAPE, curcumin or curcumin extract, cloves or cloves extract, garlic or AGE or garlic oil in form of oral recipe. The composition can be used alone or in combination with antibiotics and with or without an intravenous formula presented in the patent. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more ingredients including mixed propolis to achieve the same goals. The compositions can be administered by oral route in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, beeswax, and the like. The advantageous use of the substance mixture according to the invention consists in preparing the product or composition that will be useful to eradicate clostridia difficile infection. The formulations can include total propolis or propolis extract, such as water, alcohol or ethyl acetate extracts.

The main advantages include;

1—Natural products and available everywhere;

2—Safe and almost no side effects;

3—Synergistic effects as a Clostridia difficile colitis healer as well as an antimicrobial to eradicate intestinal infections, and

4—Provides gastro-intestinal system with excellent nutritional, anti-inflammatory and antioxidants ingredients

A suggested composition for the treatment of colitis and Clostridia difficile colitis is illustrated in, not limited to, table 12.

The invention also relates to a probiotic enhancing composition, wherein the use of mixed propolis with nigella sativa or thymoquinone or nigella sativa oil, acacia Arabica, garlic or aged garlic or garlic oil, honey, bee pollen or mixed bee pollen, olive oil or cloves or clove extract to stimulate growth of gut bacteria particularly in patients using antibiotic or in patients with colitis. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more than two of the ingredients including propolis to achieve the same goals. The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful for stimulation of beneficial gut microorganisms in normal individuals and in patients with chronic diseases, on antibiotic management or having colitis or gastrointestinal infections. The formulations can include total propolis or propolis extract, such as water, alcohol or ethyl acetate extracts.

The main advantages include:

1—Natural products and available everywhere;

2—Safe and almost no side effects;

3—Synergistic effect to stimulate beneficial gut microbial growth; and

4—Provides gastrointestinal tract with excellent nutritional, anti-inflammatory and antioxidant ingredients.

A suggested composition for enhancing gut microbiota (probiotic) is illustrated in, not limited to, table 13.

The invention also relates to an inhalation healing composition, wherein another aspect of the invention relates to use of the mixed propolis with or without honey in an inhalation composition for management of respiratory diseases. The compositions may be presented in unit-dose or multi-dose containers. And they may include additional ingredients that are conventional in the art with inhalation formulations. The compositions can be administered by inhalation in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, emulsifiers, and the like. The advantageous use of the substance mixture according to the invention consists in preparing the product or composition that will be useful for management of lower and upper respiratory tract infections, sinusitis and obstructive lung diseases. The healing herein includes curing, alleviating, reversing, palliative and prophylactic treatment of the condition. The formulations can include total propolis or propolis extract, such as water, alcohol or ethyl acetate extracts.

Honey (comprises from 10-70% w/v honey in suitable solution such as normal saline) is mixed with mixed propolis extract (comprises from 0.1-30% wt/v mixed propolis extract in suitable solution such as normal saline). The mixture can include any ratio, preferably 1:1 or 2:1 (honey/propolis). The dose could be changed to any other doses found to be more effective with lowest or no side effects. The composition offers the advantage of using ingredients of a natural origin which are readily available and whose association shows excellent high nutritional value, bronchodilator activity, wound healing property, and antioxidant, anti-inflammatory action and antimicrobial activity. Mixed propolis can be used alone without honey.

The main advantages include;

1—Natural products and available everywhere;

2—Safe and almost no side effects;

3—Synergistic effects as upper and lower respiratory system healer as well as antimicrobial to eradicate infections; and

4—Provides respiratory system with excellent nutritional, anti-inflammatory and antioxidant ingredients.

The invention also comprises the use of mixed propolis alone or a mixture of mixed propolis and honey for oral administration or parenteral administration for management of parasitic diseases. The composition may include other natural ingredients such as garlic, curcumin, nigella sativa, thymoquinone, allicin and CAPE. The composition is used for management of parasitic diseases such as, not limited to, Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total propolis or propolis extract, such as water, alcohol or ethyl acetate extracts.

The main advantages include:

1—Natural products and available everywhere;

2—Safe and almost no side effects;

3—Synergistic effects to treat parasitic infection; and

4—Provides the gastrointestinal tract with excellent nutritional, anti-inflammatory and antioxidants ingredients

Oral or parenteral compositions are used as part of management of parasitic diseases. The oral composition includes:
Honey: 0.3-2 g/kg.b.wt (or extract of honey at 0.15-1 g/kg).
Mixed propolis or their extracts: 0.02-0.14/kg.b.wt
Curcumin or curcumin extract: 0.014-0.40/kg.b.wt
Nigella sativa or its extract: 0.05-0.4/kg.b.wt
Garlic or its extract: 0.014-0.285/kg.b.wt
The extract of ingredients might include water extract, ethanol extract or ethyl acetate extracts or any other suitable extract.

The parenteral composition includes:
Honey: 0.1-2 g/k.b.wt (Honey extract can be used at 0.15-1 g/kg)
Mixed propolis extract: 0.1-30 mg/kg.b.wt
CAPE: 0.001-10 mg/kg.b.wt or 0.1-500 micromole/kg.b.wt
Thymoquinone: 0.001-5 mg/kg.b.wt
Allicin: 1-5 mg/kg.b.wt
Curcumin or curcumin extract: 0.1-40 mg/kg

The extract of ingredients might include water extract, ethanol extract or ethyl acetate extracts or any other suitable extract.

The parenteral composition is intended to be part of management of, not limited to, malaria, fluke, liver amoebiasis, trypanosome cruzi, hydatid cyst, toxoplasmosis and visceral or cutaneous leishmaniasis.

### DETAILED DESCRIPTION OF THE PREFERRED EMBODIMENTS

The invention is described below with reference to specific experiments and examples.

### Experiment 1—Effect of Mixed Propolis as an Antimicrobial

Two types of propolis collected from different geographical area (multi-plants forests) were studied; propolis A and propolis B. Alcohol extraction was performed. Both propolis were crushed after freezing with liquid nitrogen to make a powder, and then 100 g of propolis A and propolis B were added to 1 L of 70% ethyl alcohol and kept in a beaker covered with aluminum foil for 7 days at room temperature with frequent shaking. The alcohol was evaporated and the extract of propolis A and propolis B were weighed. Mixed propolis (from propolis A and propolis B extracts) was prepared by mixing equal amount of propolis A and propolis B extracts.

The extracted powder of each propolis (propolis A, B and mixed) was dissolved in 70% ethyl alcohol to make propolis concentration 5% (weight/volume) and then various concentrations were made after dilution with nutrient agar (0.05 to 2%).

In order to study the antimicrobial activity of the propolis A, B and mixed propolis on the pathogenic isolates (E. coli, S. aureus and C. albican), specimen of each pathogen was cultured in broth containing different concentrations of ethanol extract propolis A, B or mixed propolis to measure minimum inhibitory concentration. After incubation at 37° C. for 24 h, a loopful of the cultures of each of the specimen microorganisms was streaked onto agar plates, incubated aerobically at 37° C., and inspected after 24 h for microbial growth. Bacterial growth was assessed visually on solid media as: 0; no growth, 1; little growth, 2; mild growth, 3; moderate growth, and 4; heavy growth. The results showed that mixed propolis is more potent than propolis A and propolis B to eradicate the pathogens tested (Table 1)

TABLE 1
antimicrobial effects of single propolis or mixed propolis
on human pathogens. MIC = minimum inhibitory concentration.

| Type of | | | Propolis concentration % wt/v | | | | | | |
|---|---|---|---|---|---|---|---|---|---|
| propolis | Microorganism | control | 0.10 | 0.15 | 0.20 | 0.25 | 0.30 | 0.35 | MIC |
| Propolis | E. coli | 4+ | 3+ | 3+ | 0 | 0 | 0 | 0 | 0.20% |
| A | S. aureus | 4+ | 3+ | 3+ | 1+ | 0 | 0 | 0 | 0.25% |
| | C. albicans | 4+ | 4+ | 3+ | 3+ | 2+ | 0 | 0 | 0.30% |
| Propolis | E. coli | 4+ | 3+ | 2+ | 1+ | 0 | 0 | 0 | 0.25% |
| B | S. aureus | 4+ | 3+ | 3+ | 2+ | 0 | 0 | 0 | 0.25% |
| | C. albicans | 4+ | 4+ | 3+ | 3+ | 1+ | 1+ | 0 | 0.35% |
| Mixed | E. coli | 4+ | 2+ | 0 | 0 | 0 | 0 | 0 | 0.15% |
| propolis | S. aureus | 4+ | 2+ | 0 | 0 | 0 | 0 | 0 | 0.15% |
| A + B | C. albicans | 4+ | 3+ | +1 | 0 | 0 | 0 | 0 | 0.20% |

### Experiment 2—Effect of Mixed Propolis on Wound Healing

Twenty-four Swiss mice were used for experimentation. Each animal was restrained in clean and well-ventilated box. The animals were given access to water and feed at libitum. All the experiments were conducted in accordance with the internationally accepted principles for laboratory animal use and care.

Under inhalation anesthesia with use of halothane in a drop jar, 12 mm surgical incision was made on the dorsum of each mouse aseptically. The animals were divided into four groups each contain six mouse; group one; no treatment, group two; treated with propolis A applied to wound directly three times a day, group 3; treated with propolis B applied directly to wound three times daily and group four; treated with mixed propolis applied directly to the wound three times daily. Assessment of the wound was made by 12 hourly examinations of wounds for redness, edema, discharge, gapping and closure. Results showed that complete closure was obtained in 7-8 days in the control group, 5-6 days in group treated with propolis A and B and the closure of wound was obtained in 3-4 days in the animals treated with mixed propolis application.

### Safety of Mixed Propolis Extracts Intravenous Infusion

This experiment was carried out to investigate whether intravenous mixed water-soluble propolis extract or alcoholic propolis extract could elicit immediate or remote adverse effects. Two multifloral propolis (propolis A and propolis B) were collected from different geographical areas. Propolis alcoholic extract or propolis water extract were used. The extracts were mixed together in ratio 1:1 wt/wt to prepare mixed propolis extracts.

Nine sheep were used in this experiment. All the experiments were conducted in accordance with the internationally accepted principles for laboratory animal use and care. Blood investigations, including hematological and biochemical tests, were performed after 12 h of fasting. Then the animals (3 sheep) received intravenous propolis A water extract solution in a dose of 30 mg/kg.b.wt.; the other 3 sheep received intravenous propolis B water extract solution in a dose of 30 mg/kg.b.wt., and 3 sheep received intravenous mixed propolis water extract solution in a dose of 30 mg/kg.b.wt. The extract was dissolved in 100 mL of normal saline. The infusion was given over an hour. During infusion, animals were observed for any abnormal signs such as shortness of breath, shivering, allergic reaction, restlessness, or changes in the level of consciousness. Blood investigations were repeated after 5 days. The animals were kept for 1 month of observation of adverse effects. The same experiment was repeated after one month with use of the same animals whereas they received intravenous propolis ethanol extracts (propolis A, B and mixed propolis).

The results showed that no adverse effects were obtained during and after intravenous propolis A, B and mixed propolis extracts infusion. No significant changes in BUN, serum creatinine,

TABLE 2
effect of propolis A and propolis B and mixed propolis (A + B) water and ethanoic extracts on blood variables.

| | BUN | Creatinine | SGOT | SGPT | WBC | Hemoglobin | Blood sugar |
|---|---|---|---|---|---|---|---|
| Variables | (baseline) | (baseline) | (baseline) | (baseline) | (baseline) | (baseline) | (baseline) |
| Propolis | 18.2 +/− 4 | 1.1 +/− 0.2 | 44 +/− 6 | 18+/−3 | 12.4 +/− 1.5 | 11.4 +/− 2.2 | 111 +/− 12 |
| A water | (17.2 +/− 3) | (1.2 +/− 0.4) | (42 +/− 4) | (19 +/− 5.2) | (11.1 +/− 2) | (11.9 +/− 3.4) | (120 +/− 16) |
| extract | | | | | | | |
| Propolis | 18.9 +/− 4.8 | I.2 +/− 0.22 | 47 +/− 8 | 20 +/− 4.4 | 11 +/− 4 | 10.8 +/− 1.6 | 118 +/− 18 |
| A ethanol | (18.2 +/− 4) | (1.1 +/− 0.2) | (44+/− 6) | (18 +/− 3) | (12.4 +/− 1) | (11.4 +/− 2.2) | (111 +/− 12) |
| extract | | | | | | | |
| Propolis | 17.2 +/− 4 | 1.2 +/− 0.2 | 47 +/− 8 | 17 +/− 7 | 10 +/− 1 | 11 +/− 2 | 115 +/− 14 |
| B water | (18.2 +/− 4) | (1.1 +/− 0.2) | (44 +/− 6) | (18 +/− 3) | (12.4 +/− 1.5) | (11.4 +/− 2.2) | (111 +/− 12) |
| extract | | | | | | | |
| Propolis | 18 +/− 4.8 | I.2 +/− 0.22 | 49 +/− 6 | 20 +/4 | 11 +/− 6 | 10 +/− 1 | 116 +/− 18 |
| B ethanol | (17.2 +/− 4) | (1.2 +/− 0.2) | (47 +/− 8) | (17 +/− 7) | (10 +/− 1) | (11 +/− 2) | (115 +/− 14) |
| extract | | | | | | | |
| Mixed | 20.2 +/− 6 | 1.3 +/− 0.2 | 46 +/− 6 | 19 +/− 6 | 10.4 +/− 1.5 | 12.4 +/− 2 | 122 +/− 12 |
| propolis | (19.2 +/− 4) | (1.2 +/− 0.1) | (44 +/− 4) | (20 +/− 5) | (9.1 +/− 2) | (11.9 +/− 3.4) | (110 +/− 16) |
| water | | | | | | | |
| extract | | | | | | | |
| Mixed | 18.9 +/− 4 | I.2 +/− 0.4 | 49 +/− 8 | 20 +/− 4 | 11 +/− 4 | 11.8 +/− 1 | 118 +/− 18 |
| propolis | (20.2 +/− 6) | (1.3 +/− 0.2) | (46 +/− 6) | (19 +/− 6) | (10.4 +/− 1) | (12.4 +/− 2) | (122 +/− 12) |
| ethanol | | | | | | | |
| extract | | | | | | | |

SGPT, SGOT, blood sugar, white blood cell or hemoglobin were observed (Table 2).

### Preparation of Propolis Water Extract

Propolis was crushed after freezing with liquid nitrogen to make a powder, and then 100 g of propolis were added to 1L of water and 20-50 grams of soap and kept in a beaker covered with aluminum foil for 7 days at room temperature with frequent shaking. The extract was separated with use of filter paper The extract was filtered and water was evaporated and the propolis extract was weight and dissolve in water to obtain required concentration, then the water was evaporated with use of hot bath water. The propolis extract is weighed and dissolved in water to obtain the required concentration.

### Mixed Propolis in Parasitic Infection

Fifteen patients with Entamoeba histolytica, infections were divided into three treatment groups. Group 1 (5 patients) treated with propolis A (0.1 g/kg.b.wt) daily; group 2 (5 patients) treated with propolis B (0.1 g/kg.b.wt) daily, and group 3 (5 patients) were treated with mixed propolis (A+B) in similar dose (0.1 g/kg.b.wt) daily. In first and second group 3/5 patients showed no infection by 5-7 days of treatment and 5/5 patients in group three showed no infection by 4-6 days of treatment. The same experiments were repeated in other 15 patients with Giardia lamella infection and 15 patients with Trichomonas vaginalis infection. The results showed that mixed propolis was more effective than propolis A or propolis B alone.

To one skilled in the art, various modifications of the invention, without departing from the scope may include:
1—incorporating new components to optimize efficacy or commercial processing
2—altering the % weight of each constituent
3—introducing isolated chemical compounds derived from each ingredient extract individually or in combination to produce similar or better therapeutic results
4—or changing the formulation by subtracting aforementioned ingredients or using various combinations.

All of the ingredients are natural substances and have various antimicrobial, antioxidant, anti-inflammatory and nutritional properties.

### SPECIFIC EXAMPLES OF COMPOSITIONS

The tables below provide suggested compositions of mixed propolis along with other ingredients for treating various conditions

TABLE 3
Examples of wound healing compositions

| Formulae | Ingredient 1 | concentration | | Ingredient 2 | concentration | addition |
|---|---|---|---|---|---|---|
| A | Mixed propolis | 100 | grams | Bee venom or | 0.1-15 mg | |
| | or extract | | | Melittin | 0.1-5 mg | |
| B | Mixed propolis | 1-95% | | Royal jelly or | 5-30% | One or more pharmaceutically |
| | or extract | | | mixed royal jelly | | acceptable diluents or carriers can be |
| | | | | | | added to adjust the dose such as |
| | | | | | | honey |
| C | Mixed propolis | 1-96% | | Bee pollen or | 4-20% | One or more pharmaceutically |
| | or extract | | | mixed bee pollen | | acceptable diluents or carriers can be |
| | | | | | | added to adjust the dose such as |
| | | | | | | honey |
| D | Bee venom or | 0.1-15 | mg | Royal jelly or | 5-30% | One or more pharmaceutically |
| | Mellitin | 0.1-5 | mg | mixed royal jelly | | acceptable diluents or carriers can be |
| | | | | | | added to adjust the dose such as |
| | | | | | | honey |

| | | | | | |
|---|---|---|---|---|---|
| E | Bee venom or | 0.1-15 mg/100 grams | Bee pollen or | 4-20% | One or more pharmaceutically |
| | Mellitin | 0.1-5 mg/100 grams | mixed bee pollen | | acceptable diluents or carriers can be |
| | | | | | added to adjust the dose such as |
| | | | | | honey |
| F | Royal jelly or | 5-30% | Bee pollen or | 4-10% | One or more pharmaceutically |
| | mixed royal jelly | | mixed bee pollen | | acceptable diluents or carriers can be |
| | | | | | added to adjust the dose such as |
| | | | | | honey |

TABLE 4
Mixed propolis based wound healing composition. It includes mixed propolis or mixed propolis
extract alone or all the ingredients mixed together, or mixed propolis is mixed with any one
of the ingredients or mixed with two or more than two of the ingredients. One or more
pharmaceutically acceptable diluents or carriers can be added to adjust the dose such as
beeswax. Any two or more than two of the ingredients can be mixed together to prepare skin/
wound healing compositions. The compositions might include acacia Arabica 1-70%, clove or
clove oil 1-70%, garlic or aged garlic or AGE or garlic oil 1-50%, curcumin 1-80%, olive oil
1-50%, nigella sativa, thymoquinone 1-60% wt/wt, bee pollen or mixed bee pollen 4-20%, royal
jelly or mixed royal jelly, 5-20%.

| | Concentrations | | | |
|---|---|---|---|---|
| | | Ethanol or water | | |
| | | extract or ethyl | | |
| Ingredients | Crude or Powder | acetate extract | oil | Example of formulae |
| Mixed propolis | 1-100% | 1-100% | | 10% |
| CAPE | 0.1-1000 | | | CAPE 50 |
| | micromole/100 g | | | micromole/100 g |
| Honey | 10-99% | 5-49% | | 50% |
| Nigella sativa or | 0.1-15% | 0.1-15% | 0.1-10% | 5% |
| Thymoquinone | 0.1-5% | | | Or 5% thymoquinone |
| Curcumin | 5-20% | 5-10% | | 5% |
| Garlic crude or | 5-20% | 1-10% | 0.5-3% | 5% Or |
| Aged Garlic Extract | 1-15% | 1-5% | | 5% aged garlic extract |
| Olive oil | 5-10% | | | 6% |
| Bee pollen powder or mixed | 4-10% | 3-6% | | 5% |
| bee pollen | | | | |
| Clove | 1-15% | 0.5-10% | 0.1-10% | 4% |
| Bee venom or | 0.1-15 mg/100 g | 0.1-15 mg/100 g | | 0.1 mg/100 g |
| Melittin | 0.1-5 mg/100 g | 0.1-5 mg/100 g | | or melittin 0.1 mg/100 g |
| Acacia Arabica | 1-20% | 1-10% | | 4% |
| Royal jelly or mixed royal | 1-20% | 5-10% | | 6% |
| jelly | | | | |

TABLE 5
Wart healing composition; It includes mixed propolis or mixed propolis extract alone
or all the ingredients mixed together, or propolis is mixed with any one of the ingredients
or mixed with two or more than two of the ingredients, or any two or more than two
of the ingredients are mixed together. One or more pharmaceutically acceptable diluents
or carriers can be added to adjust the dose such as beeswax. Multiple compositions can
be prepared with mixing two or more ingredients. The compositions might include clove
or clove oil 1-70%, garlic or aged garlic or AGE or garlic oil 1-50%, olive oil 1-50%,
and nigella sativa or thymoquinone 1-60% wt/wt.

| | Concentrations | | | |
|---|---|---|---|---|
| | | Ethanol or water or | | |
| Ingredients | Crude or Powder | ethyl acetate extract | oil | Example of formulae |
| Mixed propolis | 1-100% | 1-100% | | 30% |
| CAPE | 1-100 | | | CAPE 50 |
| | micromole/100 g | | | micromole/100 g |
| Honey | 30-70% | 15-35% | | 40% |
| Nigella sativa or | 0.1-20% | 0.1-20% | 0.1-10% | 10% |
| Thymoquinone | 0.1-10% | | | Or 10% thymoquinone |
| Garlic crude or | 5-20% | 1-10% | 0.5-3% | 5% Or |
| Aged Garlic Extract | 1-15% | 1-5% | | 5% aged garlic extract |
| Olive oil | 5-10% | | | 5% |
| Cloves | 1-15% | 0.5-15% | 0.1-15% | 15% |

TABLE 6
Cutaneous leishmaniasis healing composition. It includes mixed propolis or mixed propolis
extract alone or all the ingredients mixed together, or propolis is mixed with any one
of the ingredient or mixed with two or more than two of the ingredient. One or more pharmaceutically
acceptable diluents or carriers can be added to adjust the dose such as bee wax. Multiple
compositions can be prepared with mixing two or more ingredients. The compositions might
include clove or clove oil 1-70%, garlic or aged garlic or AGE or garlic oil 1-50%, curcumin
1-80%, and nigella sativa or thymoquinone 1-60% wt/w

| | Concentrations | | | |
|---|---|---|---|---|
| | | Ethanol or water or | | |
| Ingredients | Crude or Powder | ethyl acetate extract | oil | Example of formulae |
| Mixed propolis | 1-100% | 1-100% | | 20% |
| CAPE | 1-1000 | | | 50 |
| | micromole/100 g | | | micromole/100 g |
| Honey | 30-70% | 15-30% | | 50% |
| Nigella sativa or | 0.1-20% | 0.1-10% | 0.1-10% | 10% |
| Thymoquinone | 0.1-10% | | | Or 10% thymoquinone |
| Curcumin | 5-20% | 5-10% | | 5% |
| Garlic crude or | 5-20% | 1-15% | 0.5-3% | 10% Or |
| Aged Garlic Extract | 1-15% | 1-10% | | 10% aged garlic extract |
| Cloves | 1-15% | 0.5-10% | 0.1-10% | 5% |

TABLE 7
Herpes healing composition. It includes mixed propolis or mixed propolis extract
alone or all the ingredients mixed together, or propolis is mixed with any one of
the ingredients or mixed with two or more than two of the ingredients, or two or
more than two of the ingredients are mixed together. One or more pharmaceutically
acceptable diluents or carriers can be added to adjust the dose such as beeswax.

| | Concentrations | | | |
|---|---|---|---|---|
| | | Ethanol or water or | | |
| Ingredients | Crude or Powder | ethyl acetate extract | oil | Example of formulae |
| Mixed propolis | 1-100% | 1-100% | | 10% |
| CAPE | 1-100 | | | 50 |
| | micromole/100 g | | | micromole/100 g |
| Honey | 30-99% | 15-49% | | 60% |
| Nigella sativa | 0.1-5% | 0.1-5% | | 5% |
| Or Thymoquinone | | | | |
| Curcumin | 5-20% | 5-10% | | 10% |
| Cloves | 1-15% | 0.5-10% | 0.1-10% | 5% |
| Bee venom or | 0.1-15 mg/100 g | 0.1-15 mg/100 g | | 0.2 mg/100 g or melittin |
| Melittin | 0.1-5 mg/100 g | 0.1-5 mg/100 g | | 0.1 mg/100 g |
| Royal jelly or mixed royal | 1-20% | 5-10% | | 6% |
| jelly | | | | |
| Garlic or AGE | 5-20% or | 1-10% | 0.5-3% | 5% |
| | AGE 1-15% | | | |

TABLE 8
Peritonitis healing composition. It includes mixed propolis
extract alone or all the ingredients mixed together, or propolis
is mixed with any one of the ingredients or mixed with two or
more than two of the ingredients. Multiple compositions including
any two or more than two of the ingredients can be prepared.
One or more pharmaceutically acceptable diluents or carriers
can be added to adjust the dose and to make the administration
of the ingredients parenterally feasible. Daily dose of the
formulae 0.5-3 gram/kg · b · wt.

| | | |
|---|---|---|
| | Doses | Example of |
| Ingredients | (per kg/day) | the formulae |

| | | | | |
|---|---|---|---|---|
| Mixed propolis extract | 0.005-0.1 | g | 1 | g |
| Curcumin extract | 0.001-0.1 | g | 1 | g |
| Honey or | 1-2 | g | 70 | g |
| Honey extract in water, | .5-1 | g | | |
| ethanol or ethyl acetate | | | | |
| Thymoquinone | 0.002-0.02 | g | 0.5 | g |
| CAPE | 0.001-0.03 | g | 0.5 | g |
| Nigella sativa extract | 0.005-0.1 | g | 1 | g |
| Or | | | | |
| Nigella sativa oil | 0.0142-0.5 | ml | 5 | ml |

TABLE 9
General oral healing formulae. It includes mixed propolis or mixed propolis
extract alone or all the ingredients mixed together, or mixed propolis is mixed
with any one of the ingredients or mixed with two or more than two of the ingredients.
One or more pharmaceutically acceptable diluents or carriers can be added to
adjust the dose. Daily dose of the formulae 0.5-3 gram/kg · b · wt.
Two or more than two ingredients can be mixed together to form multiple oral
formulations. Other ingredients of the mixtures might include date seed powder
(5-20%), why protein (5-20%), peanuts or any other type of nuts (5-20).

| | Doses per 70 kg · b · wt/day | | |
|---|---|---|---|
| | | | Example of the |
| Ingredients | Gram/kg · b · wt/day | Gram/day | formulae gram |
| Mixed propolis | 0.011-0.285 | 0.8-20 | 5 |
| Or propolis extract | 0.005-0.21 | 0.4-15 | 5 |
| CAPE | 0.001-003 | 0.07-0.21 | 0.1 |
| Honey or | 0.1-2 | 7-140 | 50 |
| Honey extract with water, ethanol | 0.5-1 | 3.5-70 | 25 |
| or ethyl acetate | | | |
| Royal jelly or mixed royal jelly | 0.014-0.85 | 1-6 | 5 |
| Nigella sativa | 0.007-0.28 | 0.5-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Bee pollen or mixed bee pollen | 0.142-0.57 | 10-40 | 15 |
| Olive oil | 0.142-0.57 | 10-40 | 20 |
| Curcumin or curcumin extract | 0.007-0.42 | 0.5-20 | 10 |
| Cloves or cloves extract | 0.007-0.14 | 0.5-10 | 3 |
| Or clove oil | 0.014-0.14 | 1-10 | or clove oil 2 ml |
| Flaxseed | 0.142-0.57 | 10-40 | 10 |
| Or flaxseed oil | 0.014-0.085 | 1-6 | 1 |
| Green tea or green tea extract | 0.007-0.071 | 0.5-5 | 1 |
| Fish oil | 0.014-0.042 | 1-3 | 2 |
| Walnuts | 0.011-0.71 | 20-50 | 20 |
| | | Or | Or |
| Or walnuts oil | 0.014-0.071 | oil 1-5 | oil 3 |
| Ginger powder | 0.014-0.285 | 1-20 | 5 |
| Or Ginger water extract | 0.014-0.285 | 1-20 | Or water extract 3 |
| Or Ginger ethanol extract | 0.0014-0.15 | 0.1-10 | Or ethanol extract 2 |
| Fenugreek seeds powder | 0.028-0.214 | 2-15 | 5 |
| Or extract | 0.004-0.028 | 0.3-2 | Or extract 1 |

TABLE 10
Kidney composition. It includes mixed propolis or mixed propolis extract alone
or all the ingredients mixed together, or propolis is mixed with any one of
the ingredients or mixed with two or more than two of the ingredients. One or
more pharmaceutically acceptable diluents or carriers can be added to adjust
the dose. Daily dose of the composition 0.5-3 gram/kg · b · wt. Two
or more than two ingredients can be mixed together to form new compositions.

| | Doses | | |
|---|---|---|---|
| | | | Example of the |
| Ingredients | Gram/kg · b · wt | Gram/day | formulae |
| Mixed propolis | 0.011-0.141 | 0.8-10 | 5 |
| Or mixed propolis extract | 0.005-0.141 | 0.4-10 | 5 |
| CAPE | 0.001-003 | 0.07-0.21 | 0.1 |
| Honey or | 0.1-2 | 7-140 | 50 |
| Extract with water, alcohol or ethyl | .05-1 | 3.5-70 | 25 |
| acetate | | | |
| Royal jelly or mixed royal jelly | 0.014-0.085 | 1-6 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.14 | 1-10 | 5 |
| Or garlic oil | 0.0014-0.071 | 0.1-5 | 0.5 |
| Bee pollen or mixed bee pollen | 0.142-0.57 | 10-40 | 20 |
| Olive oil | 0.142-0.57 | 100-40 | 30 |
| Curcumin or curcumin extract | 0.014-0.42 | 1-30 | 20 |
| Flaxseed | 0.142-0.57 | 10-40 | 10 |
| Or flaxseed oil | 0.014-0.085 | 1-6 | 1 |
| Ginger powder | 0.014-0.285 | 1-20 | 5 |
| Or ginger water extract | 0.014-0.285 | 1-20 | Or water extract 3 |
| Or ginger ethanol extract | 0.0014-0.15 | 0.1-10 | Or ethanol extract 2 |
| Green tea or green tea extract | 0.0071-0.071 | 0.5-5 | 1 |

TABLE 11
H. pylori composition includes mixed propolis or mixed propolis extract alone
or all the ingredients mixed together, or propolis is mixed with any one of the
ingredients or mixed with two or more than two of the ingredients. One or more
pharmaceutically acceptable diluents or carriers can be added to adjust the dose.
Daily dose of the composition 0.5-3 gram/kg · b · wt. Multiple compositions
can be prepared by mixing two or more than two of the ingredients.

| | Doses per 70 kg · b · wt/day | | |
|---|---|---|---|
| | | | Example of the |
| Ingredients | Gram/kg · b · wt/day | Gram/day | formulae/gram |
| Mixed propolis | 0.011-0.285 | 0.8-20 | 5 |
| Or mixed propolis extract | 0.005-0.21 | 0.4-15 | 5 |
| CAPE | 0.001-003 | 0.07-0.21 | 0.1 |
| Honey or | 0.1-2 | 7-140 | 50 |
| Extract with water, ethanol or ethyl acetate | .05-1 | | |
| Nigella sativa | 0.014-0.28 | 1-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Bee pollen or mixed bee pollen | 0.142-0.57 | 10-40 | 15 |
| Olive oil | 0.142-0.57 | 100-40 | 20 |
| Curcumin or curcumin extract | 0.014-0.42 | 1-20 | 10 |
| Cloves or cloves extract | 0.007-0.14 | 0.5-10 | 1 |
| Or clove oil | 0.014-0.14 ml | 1-10 ml | clove oil 2 ml |
| Fish oil | 0.014-0.042 | 1-3 | 2 |

TABLE 12
Clostridium difficile colitis composition. It includes mixed propolis or mixed propolis
extract alone or all the ingredients mixed together, or mixed propolis is mixed with
any one of the ingredients or mixed with two or more than two of the ingredients.
One or more pharmaceutically acceptable diluents or carriers can be added to adjust
the dose. Daily dose of the formulae 0.5-3 gram/kg · b · wt. More compositions
could be prepared by mixing two or more than two of the ingredients.

| | Doses per 70 kg · b · wt/day | | |
|---|---|---|---|
| | | | Example of the |
| Ingredients | Gram/kg · b · wt/day | Gram/day | formulae gram |
| Mixed propolis | 0.011-0.285 | 0.8-20 | 5 |
| Or mixed propolis extract | 0.005-0.21 | 0.4-15 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Honey or | 0.1-2 | 7-140 | 50 |
| extract with water, alcohol or ethyl acetate | .05-1 | 3.5-70 | 25 |
| CAPE | 0.001-003 | 0.07-0.21 | 0.1 |
| Curcumin or curcumin extract | 0.014-0.42 | 1-30 | 20 |
| Cloves or cloves extract | 0.007-0.14 | 0.5-10 | 3 |

TABLE 13
Probiotic enhancing composition includes mixed propolis or mixed propolis extract alone
or all the ingredients mixed together, or mixed propolis is mixed with any one of the
ingredients or mixed with two or more than two of the ingredients. One or more
pharmaceutically acceptable diluents or carriers can be added to adjust the dose.
Daily dose of the formulae 0.5-3 gram/kg · b · wt. Multiple compositions can be prepared by
mixing two or more than two of the ingredients not including mixed propolis.

| | Doses per 70 kg · b · wt/day | | |
|---|---|---|---|
| | | | Example of the |
| Ingredients | Gram/kg · b · wt/day | Gram/day | formulae gram |
| Mixed propolis | 0.011-0.285 | 0.8-20 | 5 |
| Or mixed propolis extract | 0.005-0.21 | 0.4-15 | 5 |
| Honey or | 0.1-2 | 21-140 | 50 |
| Extract with water, alcohol or ethyl acetate | 0.05-1 | | |
| Royal jelly or mixed royal jelly | 0.014-0.85 | 1-6 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| CAPE | 0.001-003 | 0.07-0.21 | 0.1 |
| Bee pollen or mixed bee pollen | 0.142-0.57 | 10-40 | 15 |
| Olive oil | 0.142-0.57 | 100-40 | 20 |
| Curcumin or curcumin extract | 0.014-0.42 | 1-30 | 20 |

Accordingly, while only a few embodiments of the present invention have been shown and described, it is obvious that many changes and modifications may be made thereunto without departing from the spirit and scope of the invention.

What is claimed is:

1. A composition for treatment of a medical condition, comprising a mixture of at least two different types of propolis, the two types of propolis differing from each other in at least one of the following characteristics: color, floral and plant origin, bee species, geographic region and season of harvest.

2. The composition according to claim 1, further comprising at least one of the following additional ingredients: Honey, CAPE, bee venom, melittin, royal jelly or mixed royal jelly, bee pollen or mixed bee pollen, acacia Arabica, curcumin, nigella sativa, thymoquinone, clove oil, olive oil, garlic, flaxseed or flaxseed oil, green tea, fenugreek seeds, walnuts, date seeds, whey protein and ginger.

3. The composition according to claim 1, wherein the propolis mixture contains one type of propolis in an amount of 45-55 wt % and a second type of propolis in an amount of 45-55 wt %.

4. The composition according to claim 1, wherein the composition is formulated for topical application to the skin.

5. The composition according to claim 1, wherein the composition is configured for parenteral administration.

6. The composition according to claim 1, wherein the composition is configured for inhalation.

7. The composition according to claim 1, wherein the composition is configured for oral administration.

8. The composition according to claim 4, wherein the composition is in the form of a gel, ointment, spray, suppository, liquid or cream.

9. The composition according to claim 4, wherein the composition is disposed on a surface of a dressing for wound care.

10. The composition according to claim 1, wherein the mixed propolis is combined with honey and wherein the mixed propolis is present in an amount of between 20-90 wt % and the honey is present in an amount of 10-80% wt %.

11. The composition according to claim 2, wherein the composition is a wound healing composition and contains mixed propolis or mixed propolis extract in an amount of 1-100 wt % or in an amount of 1-99 wt % and at least one of the following ingredients in proportions by weight:
CAPE: 0.1-1000 micromole/100 g
Nigella sativa or extracts of nigella sativa: 0.1-15% or 0.1-10% extract;
Thymoquinone: 0.1-5%;
Curcumin: 5-20% or curcumin extracts: 5-10%;
Garlic crude: 5-20% or as an extract: 1-15%;
Aged garlic extract 1-15% or as extract:1-5%
Olive oil: 5-10%;
Bee pollen powder (single or mixed): 4-10% or extract of bee pollen: 3-6%;
Cloves: 1-15% or extract of cloves: 0.5-10%;
Bee venom: 0.1-15 mg/100 g or extract 0.1-15 mg/100 g
Melittin : 0.1-5 mg/100 g; or extract 0.1-5 mg/100 g
Acacia Arabica: 1-20% or 1-10% as an extract; and
Royal jelly (single or mixed): 5-20% or extract: 5-10%.
Honey 10-99%: or honey extract 5-49%.

12. The composition according to claim 2, wherein the composition is configured for oral administration for treatment of kidney disease and includes mixed propolis in a dose of 0.011-0.141 gram per kilogram of body weight or mixed propolis extract 0.005-0.141 gram per kilogram of body weight and at least one of the following ingredients in the following amounts given in grams per kilogram of body weight:
CAPE: 0.001-0.003;
Honey: 0.1-2 or extract of honey with water, alcohol or ethyl acetate 0.5-1;
Royal jelly or mixed royal jelly: 0.014-0.085;
Nigella sativa: 0.014-0.28, or Thymoquinone: 0.001-0.4, or Nigella sativa oil: 0.014-0.14;
Acacia Arabica: 0.07-0.42;
Garlic: 0.014-0.285; or aged garlic extract: 0.014-0.14; or garlic oil: 0.0014-0.071;
Bee pollen or mixed bee pollen: 0.142-0.57;
Olive oil: 0.142-0.57;
Curcumin or curcumin extract: 0.014-0.42;
Flaxseed: 0.142-0.57 or flaxseed oil: 0.014-0.085;
Ginger powder: 0.014-0.285 or ginger water extract: 0.014-0.285 or ginger ethanol extract: 0.014-0.15;
Green tea or green tea extract: 0.0071-0.071.

13. The composition according to claim 2, wherein the composition is configured for intravenous administration and contains mixed propolis or extract of mixed propolis in a dose of 0.1-30 mg per kilogram of body weight, and at least one of the following ingredients in the following amounts:
Honey or an extract of honey in water, ethanol or ethyl acetate: 0.1-2 g/kg.b.wt;
CAPE: 0.001-10 mg/kg.b.wt or 0.1-500 micromole/kg.b.wt;
Thymoquinone: 0.001-5 mg/kg.b.wt; and
Curcumin or curcumin extract: 0.1-40 mg/kg.b.wt.

14. The composition according to claim 2, wherein the composition is configured for intraperitoneal administration and contains mixed propolis or extract of mixed propolis in a dose of 0.005-0.1 gram per kilogram of body weight at least one of the following ingredients in the following amounts per kilogram of body weight:
Curcumin extract: 0.001-0.1 g;
Honey: 1-2 g or honey extract in water, ethanol or ethyl acetate 0.5-1 g;
Thymoquinone: 0.002-0.02 g;
CAPE: 0.001-0.03 g;
Nigella sativa extract: 0.005-0.1 g or Nigella sativa oil 0.0142-0.5 ml.

15. A method for treating a medical condition, comprising administering to a subject in need thereof a composition comprising a mixture of at least two different types of propolis, the two types of propolis differing from each other in at least one of the following characteristics: color, floral and plant origin, bee species, geographic region and season of harvest.

16. The method according to claim 15, wherein the composition is administered by one of the following methods: orally, injection, parenterally, and topically.

17. The method according to claim 15, wherein the composition additionally contains at least one of the following ingredients: honey, CAPE, bee venom, melittin, royal jelly or mixed royal jelly, bee pollen or mixed bee pollen, acacia Arabica, curcumin, nigella sativa, thymoquinone, clove oil, olive oil, garlic, flaxseed or flaxseed oil, green tea, fish oil, fenugreek seeds, walnuts, and ginger.

18. The method according to claim 15, wherein the medical condition is one selected from the group consisting of wounds, burns, ulcers, skin infection, contact dermatitis, eczema, psoriasis, wrinkled skin, allergic reactions, herpes simplex, herpes zoster, cutaneous leishmaniasis, c. diff colitis, peritonitis, proteinuria, urinary calculus, anemia, for potentiating the effects of antibiotics and chemotherapy, dyslipidemia, diabetes mellitus, cardiovascular diseases, H. pylori gastritis or ulcers, dyspepsia, acute and chronic kidney diseases, hypertension, amelioration of toxic effects of heavy metals or chemotherapies or radio therapy, infectious diseases, sepsis, dehydration, malnutrition, sinusitis, lung obstructive diseases and malignancies.
